(19) United States
(12) Patent Application Publication
(10) Pub. No.: US 2025/0092596 A1
(43) Pub. Date: Mar. 20, 2025

(54) Fabric Heating Barrel With Graphene Heater

(71) Applicant: Xiuhua Zhao, Dongguan City (CN)

(72) Inventor: Xiuhua Zhao, Dongguan City (CN)

(21) Appl. No.: 18/471,175

(22) Filed: Sep. 20, 2023

(51) Int. Cl. D06F 58/10; A61L 2/10; A61L 2/26; D06F 34/20; D06F 34/32; D06F 58/26; D06F 103/52; D06F 105/28; G05D 23/19; H05B 1/02; H05B 3/46

(52) U.S. Cl. CPC D06F 58/10; A61L 2/10; A61L 2/26; D06F 34/20; D06F 34/32; D06F 58/26; G05D 23/19; H05B 1/0272; H05B 3/46; A61L 2202/11; A61L 2202/26; D06F 2103/52; D06F 2105/28; H05B 2203/013; H05B 2214/04

(57) ABSTRACT

A fabric heating barrel with a graphene heater is configured to heat fabrics and includes: an outer shell, serving as a heating container and a heating cavity disposed inside the outer shell. A graphene composite heating film is attached to at least one of an outer wall of the heating cavity and a bottom of the heating cavity. The graphene composite heating film is configured to heat the heating cavity. A controller and a temperature sensor are disposed between the outer shell and the heating cavity. The temperature sensor is configured to sense a temperature of the graphene composite heating film, and the controller is electrically connected to the temperature sensor and the graphene composite heating film. Due to the far-infrared and thermal radiation heating principle of the graphene, the temperature is more uniform. Compared to a traditional resistance heating member, the graphene composite heating film is more energy-efficient.

### CROSS REFERENCE TO RELATED APPLICATIONS

The present application claims the priority of Chinese patent application No. DAA0, filed on Sep. 19, 2023, and contents of which are incorporated herein by its entireties.

### TECHNICAL FIELD

The present disclosure relates to the field of fabric heating, and in particular to a fabric heating barrel with a graphene heater.

### BACKGROUND

A fabric is a flat, soft, and sheet-shaped object made by crossing, knotting, and connecting small, thin, and flexible objects. A woven fabric is made up of yarns that are crossing with each other. A knitted fabric is made up of yarns having knots. A nonwoven fabric is made up of yarns being joined with each other. A third fabric is made up of yarns that are crossing with each other and have knots.

A towel heating barrel in the art may be heated by a resistance heating member. A temperature sensor and a temperature switch are arranged to control a temperature to heat the towel to reach a preset temperature. In this way, an error of temperature control is large, a large amount of energy is wasted, and the user may have poor experience and feels less comfortable.

### SUMMARY OF THE DISCLOSURE

In order to solve the technical problems, the present disclosure provides a fabric heating barrel with a graphene heater. In the present disclosure, a graphene composite heating film is attached to an outer wall of a heating cavity to serve as a heating structure. A controller and a temperature sensor are disposed between an outer shell and the heating cavity. The temperature sensor is mounted on the outer wall of the heating cavity to sense a temperature of the graphene composite heating film. The controller is electrically connected to the temperature sensor.

Technical solutions of the embodiments of the present disclosure for solving the technical problems are as follows.

A fabric heating barrel with a graphene heater is configured to heat fabrics and includes: an outer shell of a heating container and a heating cavity disposed inside the outer shell. A graphene composite heating film is attached to an outer wall and/or a bottom of the heating cavity to serve as a heating structure.

A controller and a temperature sensor are disposed between the outer shell and the heating cavity. The temperature sensor is configured to sense a temperature of the graphene composite heating film, and the controller is electrically connected to the temperature sensor and the graphene composite heating film.

In some embodiments, the controller is configured as a controllable silicon controller. The controllable silicon controller may control the graphene composite heating film to heat. When the temperature inside the graphene composite heating film is about to reach a preset temperature, the controllable silicon controller decreases a heating power of the graphene composite heating film, instead of directly shutting down the heating of the graphene composite heating film. In this way, the temperature is more stable, and an error between the temperature and the preset temperature is small, such that a constant temperature may be reached, and the user experience may be improved.

In some embodiments, a protrusion is arranged at the bottom of the heating cavity. The graphene composite heating film is attached to a bottom outer surface of the protrusion. The graphene composite heating film is also electrically connected to the temperature sensor and the controller. By controlling heating of the graphene composite heating film, when the temperature inside the graphene composite heating film is about to reach the preset temperature, the controller may decrease the heating power of the graphene composite heating film or may control the graphene composite heating film to stop heating.

In some embodiments, the controller is respectively electrically connected to the graphene composite heating film attached to the outer wall of the heating cavity, the graphene composite heating film attached to the bottom of the heating cavity, and the graphene composite heating film attached to the bottom outer surface of the protrusion. The controller decrease heating powers of the graphene composite heating films disposed at the three locations or controls the graphene composite heating films disposed at the three locations to stop heating.

In some embodiments, a keypad is embedded into the outer shell. The temperature and a heating time length may be set by the keypad. A touch screen is disposed at a side of the keypad. The touch screen is configured to display temperatures of various locations of a wall of the barrel. The user may observe heating temperatures of the graphene composite heating films disposed at various locations. The touch screen is electrically connected to the controller.

According to the above technical solution, the towel or fabric is placed on the inner wall of the outer shell. The heating temperature and the heating time length may be set by the keypad. Subsequently, heating is performed. The heating is achieved by the graphene composite heating film. The heating temperature and the temperature of the graphene composite heating film can be detected in real time by the temperature sensor, and the detected temperatures are displayed on the touch screen.

In some embodiments, the controller and the temperature sensor are mounted on the outer wall or the bottom of the heating cavity.

In some embodiments, a sterilization lamp is embedded in the inner wall of the heating cavity. The sterilization lamp is a UV sterilization lamp and is configured to sterilize the towel that is being heated.

In some embodiments, a cover plate is arranged at a top of the outer shell. A support is arranged at a bottom of the outer shell. A handle is arranged on a top of the cover plate. A sealing gasket is arranged at a location where the outer shell is connected to the cover plate. By arranging the sealing gasket, the cover plate and the outer shell may be connected with each other tightly. The surface of the cover plate is arranged with the handle. The surface of the cover plate on which of the handle is arranged defines a ventilation hole. While heating, the ventilation hole defined in the cover plate may allow air ventilation, preventing the air pressure inside the barrel from being high, and the barrel may not be damaged.

According to the above technical solution, while heating, air ventilation is achieved through a ventilation hole defined in the cover plate, such that an air pressure inside the cover plate may not be high, and the device may not be damaged.

In some embodiments, a water collection tray is arranged below the heating cavity and on the inner wall of the outer shell. A lower surface of the heating cavity defines a water collection-outlet hole. Alternatively, the water collection tray is disposed below the water collection-outlet hole. In this way, after the towel or the fabric is heated or dried, water inside the heating cavity may flow through the water collection-outlet hole to be received by water collection tray or may flow through the water collection-outlet hole to enter the drain pipe to be discharged. Alternatively, both the drain pipe and the water collection tray may be arranged for discharging the water.

Advantages of the present application are as follows:

The graphene composite heating film is attached to the outer wall of the heating cavity to serve as the heating structure. The controller and the temperature sensor are disposed between the outer shell and the heating cavity. The temperature sensor is configured to sense the temperature of the graphene composite heating film. The controller is electrically connected to the temperature sensor and the graphene composite heating film. Due to the far-infrared and thermal radiation heating principle of the graphene, the temperature is more uniform. Compared to a traditional resistance heating member, the graphene composite heating film is more energy-efficient.

The controller may be a controllable silicon controller. When the temperature is about to reach the preset temperature, the controllable silicon controller may reduce a heating power of the graphene composite heating film, instead of directly shutting down heating of the graphene composite heating film. In this way, the temperature is more stable, an error between the temperature and the preset temperature is small, such that a constant temperature may be achieved, improving user experience.

In addition, the graphene composite heating film may be disposed at various locations, such as being attached to the outer wall of the heating cavity, a bottom of the heating cavity, and the outer surface of the bottom of the protrusion. In this way, heating powers at various locations may be controlled respectively, and a heating efficiency and a drying efficiency may be improved.

### BRIEF DESCRIPTION OF THE DRAWINGS

The present disclosure is described in more details below by referring to the accompanying drawings and embodiments.

FIG. 1 is a structural schematic view of the entire fabric heating barrel with a graphene heater according to a first embodiment of the present disclosure.

FIG. 2 is a front view of the fabric heating barrel with the graphene heater according to the first embodiment of the present disclosure.

FIG. 3 is a top view of the fabric heating barrel with the graphene heater according to the first embodiment of the present disclosure.

FIG. 4 is a bottom view of the fabric heating barrel with the graphene heater according to the first embodiment of the present disclosure.

FIG. 5 is a cross-sectional view of the fabric heating barrel with the graphene heater according to the first embodiment of the present disclosure.

FIG. 6 is a structural schematic view of the entire fabric heating barrel with the graphene heater according to a second embodiment of the present disclosure.

FIG. 7 is a cross-sectional view of the fabric heating barrel with the graphene heater according to the second embodiment of the present disclosure.

FIG. 8 is a structural schematic view of an interior of the fabric heating barrel with the graphene heater according to the second embodiment of the present disclosure.

Reference numerals are as follows:

1, outer shell; 2, cover plate; 3, touch screen; 4, support; 5, power cord; 6, ventilation hole; 7, graphene composite heating film; 8, protrusion; 9, controller; 10, temperature sensor; 11, current connector; 12, keypad; 13, heating cavity; 14, sterilization lamp; 15, drain pipe; 16, water collection-outlet hole; 17, water collection tray.

### DETAILED DESCRIPTION

In order to solve technical problems in the art, embodiments of the present disclosure provide a fabric heating barrel with a graphene heater. In the present disclosure, a graphene composite heating film is attached to an outer wall of a heating cavity to serve as a heating structure. A controller and a temperature sensor are disposed between an outer shell and the heating cavity. The temperature sensor is mounted on the outer wall of the heating cavity and senses a temperature of the graphene composite heating film. The controller is electrically connected to the temperature sensor. The controller may be configured as a controllable silicon controller. Due to the far-infrared and thermal radiation heating principle of the graphene of the graphene composite heating film, and due to the heating being controlled by the controller, when the temperature is about to reach the preset temperature, the controller may reduce a heating power of the graphene composite heating film, but may not directly shut down the heating of the graphene composite heating film. In this way, the temperature is more stable, an error of the temperature and the preset temperature is small, and the user experience is improved. In addition, the graphene composite heating film may be attached to the outer wall of the heating cavity and an inner wall of a protrusion, such that a heating efficiency and a drying efficiency are improved.

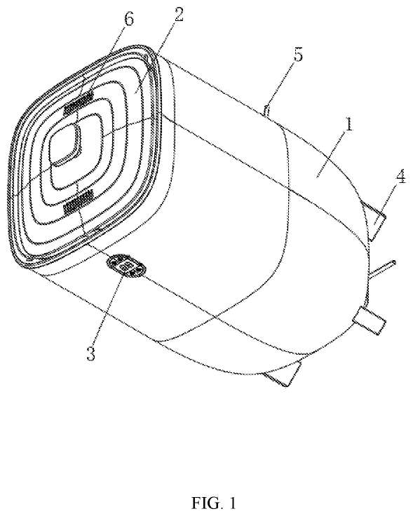

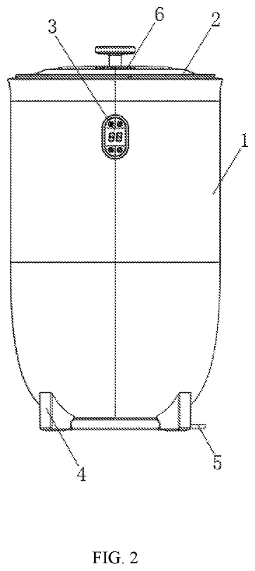

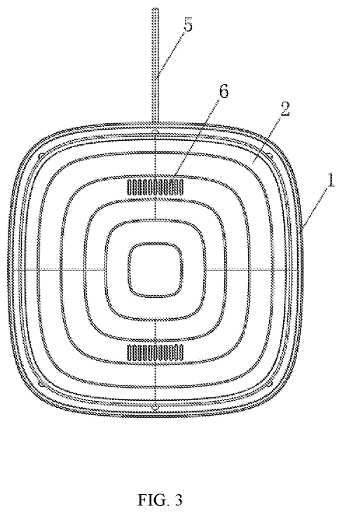

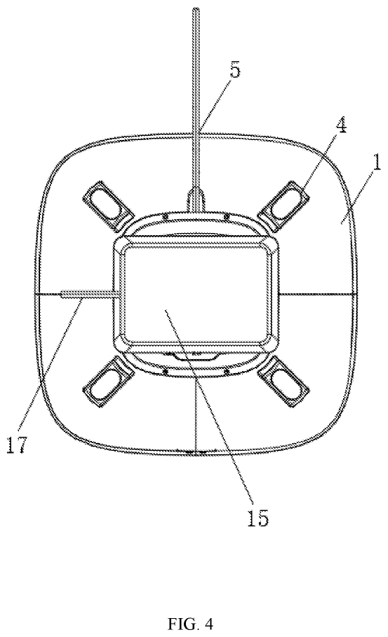

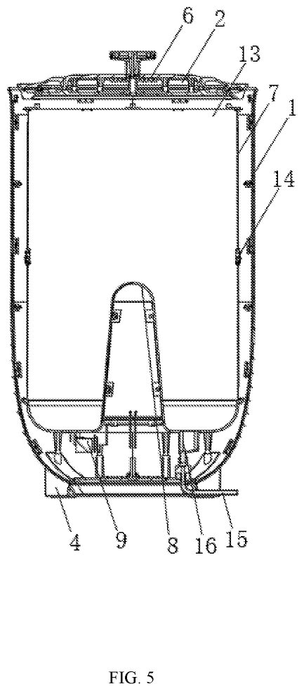

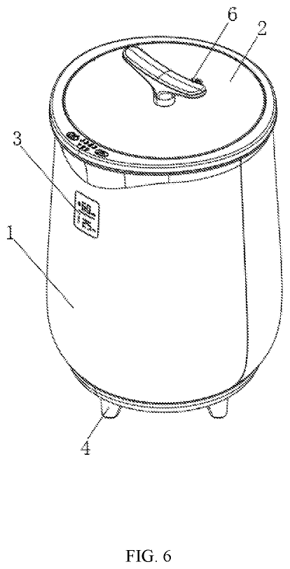

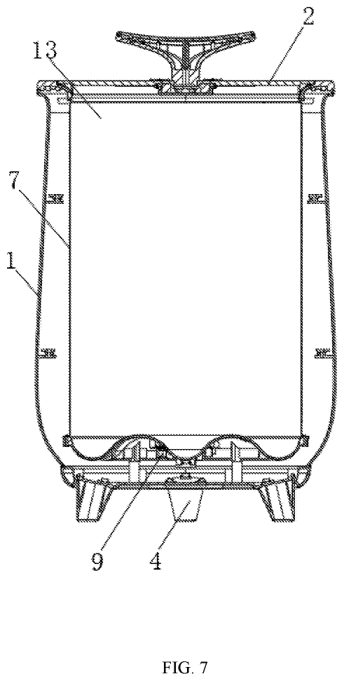

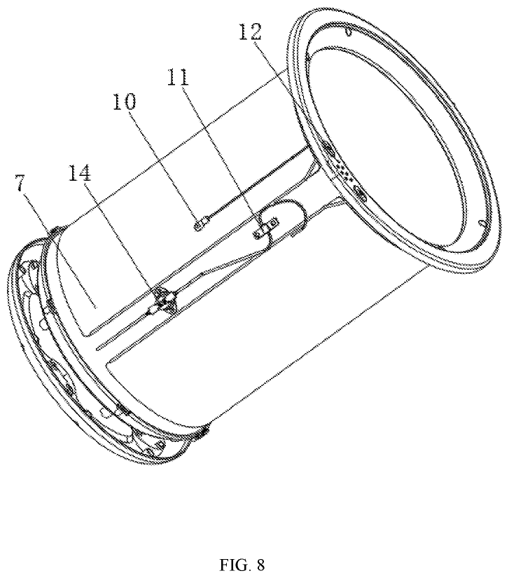

The present embodiment provides a specific structure of a fabric heating barrel with a graphene heater, as shown in FIGS. 1-8. The structure may be applied to heat fabrics. The fabric heating barrel with the graphene heater includes an outer shell 1 of a heating container and a heating cavity 13 disposed inside the outer shell 1. A graphene composite heating film 7 is attached to an outer wall or a bottom of the heating cavity 13 (alternatively, the graphene composite heating film 7 is attached to both the outer wall and the bottom of the heating cavity 13). Therefore, the graphene composite heating film 7 serves as a heating structure.

A controller 9 and a temperature sensor 10 are disposed between the outer shell 1 and the heating cavity 13. The controller 9 and the temperature sensor 10 are mounted on the outer wall or the bottom of the heating cavity 13 and sense a temperature of the graphene composite heating film 7. The controller 9 is electrically connected to the temperature sensor 10 and the graphene composite heating film 7.

The controller 9 may be configured as a controllable silicon controller. Specifically, the controller 9 is disposed at a bottom of the outer shell 1 and at an interior of the outer shell 1. Since graphene of the graphene composite heating film 7 has the far-infrared and thermal radiation heating principle, and since the controller 9 may control the heating, when the temperature inside the graphene composite heating film 7 is about to reach a preset temperature, the controller 9 decreases a heating power of the graphene composite heating film 7, instead of directly shutting down the heating of the graphene composite heating film 7. In this way, the temperature is more stable, and an error between the temperature and the preset temperature is small, such that a constant temperature may be reached, and the user experience may be improved.

In some embodiments, a protrusion 8 is arranged at the bottom of the heating cavity 13. The graphene composite heating film 7 is attached to a bottom outer surface of the protrusion 8. The graphene composite heating film 7 is also electrically connected to the temperature sensor 10 and the controller 9. Heating of the graphene composite heating film 7 is controlled by the controllable silicon controller. When the temperature inside the graphene composite heating film 7 is about to reach the preset temperature, the controllable silicon controller may decrease the heating power of the graphene composite heating film 7.

In some embodiments, the controller 9 is respectively electrically connected to the graphene composite heating film 7 attached to the outer wall of the heating cavity 13, the graphene composite heating film 7 attached to the bottom of the heating cavity 13, and the graphene composite heating film 7 attached to the bottom outer surface of the protrusion 8. The controller 9 decrease heating powers of the graphene composite heating films 7 disposed at the three locations or controls the graphene composite heating films 7 disposed at the three locations to stop heating.

Of course, the user may replace the controllable silicon controller with a relay. When the temperature of the graphene composite heating films 7 reaches the preset temperature or is about to reach the preset temperature, the relay is configured to directly shut down a heating function of the graphene composite heating film 7.

A keypad 12 is embedded into the outer shell 1. The temperature and a heating time length may be set by the keypad 12. A touch screen 3 is disposed at a side of the keypad 12. The touch screen 3 is configured to display temperatures of various locations of a wall of the barrel. The user may observe heating temperatures of the graphene composite heating films 7 disposed at various locations (including: the outer wall of the heating cavity 13, the bottom of the heating cavity 13, and the bottom outer surface of the protrusion 8). The graphene composite heating films 7 are electrically connected to the controller 9. A power cord 5 is disposed at a side of the outer shell 1. The controller 9 is electrically connected to the power cord 5.

In some embodiments, the temperature sensor may detect in real-time the heating temperatures of the graphene composite heating films disposed at various locations (including: the outer wall of the heating cavity 13, the bottom of the heating cavity 13, and the bottom outer surface of the protrusion 8) and transmit the heating temperatures to the touch screen to be displayed. Depending on situations, the user may operate the keypad 12 and takes the controller 9 to reduce the heating powers of the graphene composite heating films disposed at various locations or to control the graphene composite heating films to stop heating.

In some embodiments, a sterilization lamp 14 is embedded in an inner wall of the heating cavity 13. The sterilization lamp is a UV sterilization lamp to sterilize a towel that is being heated.

A support 4 is arranged at an end of the outer shell 1 away from the cover plate 2. In total, 4 supports are arranged. By arranging the four supports 4, the outer shell 1 is supported by the supports 4. A sealing gasket is arranged at a location where the outer shell 1 is connected to the cover plate 2. By arranging the sealing gasket, the cover plate 2 and the outer shell 1 may be connected with each other tightly. A handle is arranged on a surface of the cover plate 2. The surface of the cover plate 2 on which of the handle is arranged defines a ventilation hole 6. While heating, the ventilation hole 6 defined in the cover plate 2 may allow air ventilation, preventing the air pressure inside the barrel from being high, and the barrel may not be damaged.

In some embodiments, a water collection tray 17 is arranged below the heating cavity 13 and on the inner wall of the outer shell 1. A lower surface of the heating cavity 13 defines a water collection-outlet hole 16. A drain pipe 15 is arranged at and communicated with a bottom end of the water collection-outlet hole 16. Alternatively, the water collection tray 17 is disposed below the water collection-outlet hole 16. In this way, after the towel or the fabric is heated or dried, water inside the heating cavity 13 may flow through the water collection-outlet hole 16 to be received by water collection tray 17 or may flow through the water collection-outlet hole 16 to enter the drain pipe 15 to be discharged. The drain pipe 15 has a certain length and may be communicated with a downcomer 15.

The graphene composite heating film is attached to the outer wall of the heating cavity to serve as the heating structure. The controller and the temperature sensor are disposed between the outer shell and the heating cavity. The temperature sensor senses the temperature of the graphene composite heating film. The controller is electrically connected to the temperature sensor and the graphene composite heating film. Due to the far-infrared and heat radiation heating principle of the graphene, the temperature is more uniform. Compared to the traditional resistance heating member, the graphene composite heating film is more energy-efficient.

The controller may be configured as the controllable silicon controller. Due to the far-infrared and heat radiation heating principle of the graphene composite heating film, when the temperature is about to reach the preset temperature, the controllable silicon controller may reduce the heating power of the graphene composite heating film, instead of directly shutting down heating of the graphene composite heating film. In this way, the temperature is more stable, the error between the temperature and the preset temperature is small, such that the user experience is improved.

In addition, the graphene composite heating film may be disposed at various locations, such as being attached to the outer wall of the heating cavity, the bottom of the heating cavity, and the bottom outer surface of the protrusion. In this way, heating powers at various locations may be controlled respectively, and the heating efficiency and the drying efficiency may be improved.

At last, obviously, the above embodiments are merely examples for the purpose of clearly illustrating the present disclosure only, and are not limitation of the present disclosure. Any ordinary skilled person in the art may perform various changes or variations based on the above description. It is neither necessary nor possible to list all of potential embodiments herein. The obvious changes or variations derived therefrom shall still be within the scope of the present disclosure.

What is claimed is:

1. A fabric heating barrel with a graphene heater, configured to heat fabrics and comprising:
an outer shell, serving as a heating container;
a heating cavity, disposed inside the outer shell;
a graphene composite heating film, attached to at least one of an outer wall of the heating cavity and a bottom of the heating cavity, wherein the graphene composite heating film is configured to heat the heating cavity;
a controller and a temperature sensor, disposed between the outer shell and the heating cavity, wherein the temperature sensor is configured to sense a temperature of the graphene composite heating film, and the controller is electrically connected to the temperature sensor and the graphene composite heating film.

2. The fabric heating barrel with the graphene heater according to claim 1, wherein the controller is configured as a controllable silicon controller.

3. The fabric heating barrel with the graphene heater according to claim 1, wherein a protrusion is arranged at the bottom of the heating cavity and protrudes towards an interior of the heating cavity.

4. The fabric heating barrel with the graphene heater according to claim 1, wherein a keypad (12) is embedded into the outer shell and disposed at a top of the outer shell, a touch screen is disposed at a side of the keypad, and the touch screen is electrically connected to the controller.

5. The fabric heating barrel with the graphene heater according to claim 1, wherein the controller and the temperature sensor are mounted on the outer wall or the bottom of the heating cavity.

6. The fabric heating barrel with the graphene heater according to claim 5, wherein a sterilization lamp is embedded in an inner wall of the heating cavity.

7. The fabric heating barrel with the graphene heater according to claim 1, wherein a cover plate is disposed at a top of the outer shell, a support is disposed at a bottom of the outer shell, a handle is disposed at a top of the cover plate, and the cover plate defines a ventilation hole.

8. The fabric heating barrel with the graphene heater according to claim 1, wherein a lower surface of the heating cavity defines a water collection-outlet hole;
at least one of a drain pipe and a water collection tray is arranged, wherein,
when the drain pipe is arranged, the drain pipe is disposed at a bottom end of the water collection-outlet hole and is communicated with water collection-outlet hole;
when the water collection tray is arranged, the water collection tray is disposed below the water collection-outlet hole.
